(19) United States
(12) Patent Application Publication
(10) Pub. No.: US 2026/0188432 A1
(43) Pub. Date: Jul. 2, 2026

(54) METHOD FOR DESIGNING mRNA VACCINE, METHOD FOR PREDICTING TRANSLATION EFFICIENCY OF mRNA VACCINE, AND HARDWARE APPARATUS

(71) Applicant: IUCF-HYU (INDUSTRY-UNIVERSITY COOPERATION FOUNDATION HANYANG UNIVERSITY), Seoul (KR)

(72) Inventors: Jin-Wu NAM, Seoul (KR); Seokju PARK, Seoul (KR); Sol Been KIM, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 19/544,084

(22) Filed: Feb. 19, 2026

Related U.S. Application Data
(63) Continuation of application No. PCT/KR2024/012535, filed on Aug. 22, 2024.

(30) Foreign Application Priority Data
Aug. 23, 2023 (KR) 10-2023-0110772

(51) Int. Cl. G16B 40/00; G16B 30/00

(52) U.S. Cl. CPC G16B 40/00; G16B 30/00

(57) ABSTRACT

A method for designing an mRNA vaccine includes receiving sequence information of a candidate mRNA vaccine, extracting an input sequence including a 25nt primer-binding sequence in a 5′ UTR (Untranslated Region) of the sequence information, a 50nt sequence of the 5′ UTR immediately before a CDS (Coding sequence) region, and a 30nt sequence after a start codon of a coding region, calculating secondary structure information for the input sequence, predicting translation efficiency of the candidate mRNA vaccine by inputting the input sequence and the secondary structure information into a pre-trained deep learning model, and, when the translation efficiency of the candidate mRNA vaccine is equal to or greater than a threshold, generating a final mRNA vaccine sequence by linking a sequence of an antigen protein to the sequence information of the candidate mRNA vaccine.

### CROSS REFERENCE TO RELATED APPLICATIONS

This application is a Continuation of PCT International Application No. PCT/KR2024/012535, filed on Aug. 22, 2024, which claims priority to Korean Patent Application No. 10-2023-0110772, filed on Aug. 23, 2023, which are all hereby incorporated by reference in their entirety.

### BACKGROUND

### Technical Field

The present disclosure relates to methods and apparatus for predicting translation efficiency of an mRNA (messenger Ribonucleic Acid) vaccine.

### Description of the Related Art

Traditionally, a vaccine refers to a biological preparation containing an antigen obtained by appropriately processing a pathogen itself, a portion of its components, or a toxin. After COVID-19, an mRNA vaccine, which is a next-generation vaccine, has been commercialized. An mRNA vaccine is composed of mRNA carrying a sequence encoding an antigen and a lipid nanoparticle (LNP) carrier. The LNP of an mRNA vaccine injected into a living body delivers the mRNA carrying the sequence encoding the antigen into cells, and the cells produce the antigen protein. For an mRNA vaccine to induce an immune response, a sufficient amount of antigen must be produced. Therefore, it is important that the mRNA vaccine has high stability and translation efficiency after being administered into a living body.

The description of the related art should not be assumed to be prior art merely because it is mentioned in or associated with this section. The description of the related art includes information that describes one or more aspects of the subject technology, and the description in this section does not limit the invention.

### SUMMARY

In one or more aspects of the present disclosure, a method for predicting translation efficiency of an mRNA vaccine includes receiving, by a hardware apparatus, sequence information of a candidate mRNA vaccine, calculating, by the hardware apparatus, a partial region of a 5′ UTR (Untranslated Region) of the sequence information and secondary structure information formed by the partial region, and predicting, by the hardware apparatus, translation efficiency of the candidate mRNA vaccine by inputting the partial region and the secondary structure information into a pre-trained deep learning model.

In one or more aspects of the present disclosure, a method for predicting translation efficiency of an mRNA vaccine includes receiving, by a hardware apparatus, sequence information of a candidate mRNA vaccine, extracting, by the hardware apparatus, an input sequence including a 25 nt sequence to which a primer binds in a 5′ UTR (Untranslated Region) of the sequence information, a 50 nt sequence of the 5′ UTR immediately before a CDS (Coding sequence) region, and a 30 nt sequence after a start codon of a coding region, calculating, by the hardware apparatus, secondary structure information for the input sequence, and predicting, by the hardware apparatus, translation efficiency of the candidate mRNA vaccine by inputting the input sequence and the secondary structure information into a pre-trained deep learning model.

In one or more aspects of the present disclosure, a hardware apparatus for predicting translation efficiency of an mRNA vaccine includes an interface device configured to receive sequence information of a candidate mRNA vaccine, a storage device configured to store a pre-trained deep learning model, and a processor configured to predict translation efficiency of the candidate mRNA vaccine by inputting a partial region of a 5′ UTR (Untranslated Region) of the sequence information and secondary structure information formed by the partial region into the deep learning model.

Additional features, advantages, and aspects of the present disclosure are set forth in part in the description that follows and in part will become apparent from the present disclosure or may be learned by practice of the inventive concepts provided herein. Other features, advantages, and aspects of the present disclosure may be realized and attained by the descriptions provided in the present disclosure, or derivable therefrom, and the claims hereof as well as the drawings. It is intended that all such features, advantages, and aspects be included within this description, be within the scope of the present disclosure, and be protected by the following claims. Nothing in this section should be taken as a limitation on those claims. Further aspects and advantages are discussed below in conjunction with embodiments of the present disclosure.

It is to be understood that both the foregoing description and the following description of the present disclosure are examples, and are intended to provide further explanation of the disclosure as claimed.

### BRIEF DESCRIPTION OF THE DRAWINGS

The accompanying drawings, which are included to provide a further understanding of the present disclosure, are incorporated in and constitute a part of this present disclosure, illustrate aspects and embodiments of the present disclosure, and together with the description serve to explain principles and examples of the disclosure. In the drawings:

FIG. 1 is an example of an mRNA structure.

FIG. 2 is an example of a system for predicting translation efficiency of an mRNA vaccine.

FIG. 3 illustrates a relationship between an interaction of a 5′ UTR and a CDS sequence of an mRNA and translation efficiency.

FIG. 4 illustrates an example of input data and an encoding process of a deep learning model.

FIG. 5 is an example of a process of building a deep learning model for predicting translation efficiency of a candidate mRNA vaccine.

FIG. 6 is an example of a deep learning model for predicting translation efficiency of an mRNA vaccine.

FIG. 7 illustrates a result of evaluating performance of translation efficiency prediction of the built deep learning model.

FIG. 8 illustrates a result of comparing performance of deep learning models using various input data.

FIG. 9 is an example of an analysis apparatus for predicting translation efficiency of an mRNA vaccine.

Throughout the drawings and the detailed description, unless otherwise described, the same drawing reference numerals should be understood to refer to the same elements, features, and structures. The sizes of regions and elements, and depiction thereof may be exaggerated for clarity, illustration, and/or convenience.

### DETAILED DESCRIPTION

The following detailed description is provided to assist the reader in gaining a comprehensive understanding of the methods, apparatuses, and/or systems described herein. Accordingly, various changes, modifications, and equivalents of the systems, apparatuses and/or methods described herein will be understood by those of ordinary skill in the art.

Moreover, descriptions of well-known functions and constructions may be omitted for increased clarity and conciseness. Further, repetitive descriptions may be omitted for brevity. The progression of processing steps and/or operations described is a non-limiting example.

The sequence of steps and/or operations is not limited to that set forth herein and may be changed to occur in an order that is different from an order described herein, with the exception of steps and/or operations necessarily occurring in a particular order. In one or more examples, two operations in succession may be performed substantially concurrently, or the two operations may be performed in a reverse order or in a different order depending on a function or operation involved.

Unless stated otherwise, like reference numerals may refer to like elements throughout even when they are shown in different drawings. Unless stated otherwise, the same reference numerals may be used to refer to the same or substantially the same elements throughout the specification and the drawings. In one or more aspects, identical elements (or elements with identical names) in different drawings may have the same or substantially the same functions and properties unless stated otherwise. Names of the respective elements used in the following explanations are selected only for convenience and may be thus different from those used in actual products.

Advantages and features of the present disclosure, and implementation methods thereof, are clarified through the embodiments described with reference to the accompanying drawings. The present disclosure may, however, be embodied in different forms and should not be construed as limited to the embodiments set forth herein. Rather, these embodiments are examples and are provided so that this disclosure may be thorough and complete to assist those skilled in the art to understand the inventive concepts without limiting the protected scope of the present disclosure.

Shapes, dimensions (e.g., sizes, lengths, locations, and areas), proportions, ratios, numbers, the number of elements, and the like disclosed herein, including those illustrated in the drawings, are merely examples, and thus, the present disclosure is not limited to the illustrated details. It is, however, noted that the relative dimensions of the components illustrated in the drawings are part of the present disclosure.

When the term “comprise,” “have,” “include,” “contain,” “constitute,” “made of,” “formed of,” “composed of,” or the like is used with respect to one or more elements (e.g., components, structures, groups, circuits, networks, members, parts, areas, portions, integers, steps, operations, and/or the like), one or more other elements may be added unless a term such as “only” or the like is used. The terms used in the present disclosure are merely used in order to describe particular example embodiments, and are not intended to limit the scope of the present disclosure. The terms of a singular form may include plural forms unless the context clearly indicates otherwise. For example, an element may be one or more elements. An element may include a plurality of elements. The word “exemplary” is used to mean serving as an example or illustration. Embodiments are example embodiments. Aspects are example aspects. In one or more implementations, “embodiments,” “examples,” “aspects,” and the like should not be construed to be preferred or advantageous over other implementations. An embodiment, an example, an example embodiment, an aspect, or the like may refer to one or more embodiments, one or more examples, one or more example embodiments, one or more aspects, or the like, unless stated otherwise. Further, the term “may” encompasses all the meanings of the term “can.”

In one or more aspects, unless explicitly stated otherwise, an element, feature, or corresponding information (e.g., a level, range, dimension, or the like) is construed to include an error or tolerance range even where no explicit description of such an error or tolerance range is provided. An error or tolerance range may be caused by various factors (e.g., process factors, internal or external impact, noise, or the like). In interpreting a numerical value, the value is interpreted as including an error range unless explicitly stated otherwise.

When a positional relationship between two elements (e.g., components, structures, groups, circuits, networks, members, parts, areas, portions, and/or the like) are described using any of the terms such as “adjacent to,” “beside,” “next to,” and/or the like indicating a position or location, one or more other elements may be located between the two elements unless a more limiting term, such as “immediate(ly),” “direct(ly),” or “close(ly),” is used. Furthermore, the spatially relative terms such as the foregoing terms as well as other terms such as “column,” “row,” “vertical,” “horizontal,” “diagonal,” and the like refer to an arbitrary frame of reference.

In describing a temporal relationship, when the temporal order is described as, for example, “after,” “following,” “subsequent,” “next,” “before,” “preceding,” “prior to,” or the like, a case that is not consecutive or not sequential may be included and thus one or more other events may occur therebetween, unless a more limiting term, such as “just,” “immediate(ly),” or “direct(ly),” is used.

It is understood that, although the terms “first,” “second,” and the like may be used herein to describe various elements (e.g., components, structures, groups, circuits, networks, members, parts, areas, portions, and/or the like), these elements should not be limited by these terms, for example, to any particular order, precedence, or number of elements. These terms are used only to distinguish one element from another. For example, a first element may denote a second element, and, similarly, a second element may denote a first element, without departing from the scope of the present disclosure. Furthermore, the first element, the second element, and the like may be arbitrarily named according to the convenience of those skilled in the art without departing from the scope of the present disclosure. For clarity, the functions or structures of these elements (e.g., the first element, the second element, and the like) are not limited by ordinal numbers or the names in front of the elements. Further, a first element may include one or more first elements. Similarly, a second element or the like may include one or more second elements or the like.

In describing elements of the present disclosure, the terms “first,” “second,” “A,” “B,” “(a),” “(b),” or the like may be used. These terms are intended to identify the corresponding element(s) from the other element(s), and these are not used to define the essence, basis, order, or number of the elements.

The expression that an element (e.g., component, structure, group, circuit, network, member, part, area, portion, and/or the like) “is engaged” with another element may be understood, for example, as that the element may be either directly or indirectly engaged with the another element. The term “is engaged” or similar expressions may refer to a term such as “is connected,” “is coupled,” “is combined,” “is linked,” “is provided,” “interacts,” or the like. The engagement may involve one or more intervening elements disposed or interposed between the element and the another element, unless otherwise specified.

The terms such as a “line” or “direction” should not be interpreted only based on a geometrical relationship in which the respective lines or directions are parallel, perpendicular, diagonal, or slanted with respect to each other, and may be meant as lines or directions having wider directivities within the range within which the components of the present disclosure may operate functionally.

The term “at least one” should be understood as including any and all combinations of one or more of the associated listed items. For example, each of the phrases “at least one of a first item, a second item, or a third item” and “at least one of a first item, a second item, and a third item” may represent (i) a combination of items provided by two or more of the first item, the second item, and the third item or (ii) only one of the first item, the second item, or the third item. Further, at least one of a plurality of elements can represent (i) one element of the plurality of elements, (ii) some elements of the plurality of elements, or (iii) all elements of the plurality of elements. Further, “at least some,” “at least some portions,” “at least some parts,” “at least a portion,” “at least one or more portions,” “at least a part,” “at least one or more parts,” “at least some elements,” “one or more,” or the like of a plurality of elements can represent (i) one element of the plurality of elements, (ii) a portion (or a part) of the plurality of elements, (iii) one or more portions (or parts) of the plurality of elements, (iv) multiple elements of the plurality of elements, or (v) all of the plurality of elements. Moreover, “at least some,” “at least some portions,” “at least some parts,” “at least a portion,” “at least one or more portions,” “at least a part,” “at least one or more parts,” or the like of an element can represent (i) a portion (or a part) of the element, (ii) one or more portions (or parts) of the element, or (iii) the element, or all portions of the element.

The expression of a first element, a second elements “and/or” a third element should be understood as one of the first, second and third elements or as any or all combinations of the first, second and third elements. By way of example, A, B and/or C may refer to only A; only B; only C; any of A, B, and C (e.g., A, B, or C); some combination of A, B, and C (e.g., A and B; A and C; or B and C); or all of A, B, and C. Furthermore, an expression “A/B” may be understood as A and/or B. For example, an expression “A/B” may refer to only A; only B; A or B; or A and B.

In one or more aspects, the terms “between” and “among” may be used interchangeably simply for convenience unless stated otherwise. For example, an expression “between a plurality of elements” may be understood as among a plurality of elements. In another example, an expression “among a plurality of elements” may be understood as between a plurality of elements. In one or more examples, the number of elements may be two. In one or more examples, the number of elements may be more than two. Furthermore, when an element is referred to as being “between” at least two elements, the element may be the only element between the at least two elements, or one or more intervening elements may also be present.

In one or more aspects, the phrases “each other” and “one another” may be used interchangeably simply for convenience unless stated otherwise. For example, an expression “different from each other” may be understood as being different from one another. In another example, an expression “different from one another” may be understood as being different from each other. In one or more examples, the number of elements involved in the foregoing expression may be two. In one or more examples, the number of elements involved in the foregoing expression may be more than two.

In one or more aspects, the phrases “one or more among” and “one or more of” may be used interchangeably simply for convenience unless stated otherwise.

The term “or” means “inclusive or” rather than “exclusive or.” That is, unless otherwise stated or clear from the context, the expression that “x uses a or b” means any one of natural inclusive permutations. For example, “a or b” may mean “a,” “b,” or “a and b.” For example, “a, b or c” may mean “a,” “b,” “c,” “a and b,” “b and c,” “a and c,” or “a, b and c.”

A phrase “substantially the same” may indicate a degree of being considered as being equivalent to each other taking into account minute differences due to errors in the manufacturing or operating process.

Features of various embodiments of the present disclosure may be partially or entirely coupled to or combined with each other, may be technically associated with each other, and may be variously operated, linked or driven together in various ways. Embodiments of the present disclosure may be implemented or carried out independently of each other or may be implemented or carried out together in a co-dependent or related relationship. In one or more aspects, the components of each apparatus and device according to various embodiments of the present disclosure are operatively coupled and configured.

The terms used herein have been selected as being general in the related technical field; however, there may be other terms depending on the development and/or change of technology, convention, preference of technicians, and so on. Therefore, the terms used herein should not be understood as limiting technical ideas, but should be understood as examples of the terms for describing example embodiments.

Further, in a specific case, a term may be arbitrarily selected by an applicant, and in this case, the detailed meaning thereof is described herein. Therefore, the terms used herein should be understood based on not only the name of the terms, but also the meaning of the terms and the content hereof.

In the following description, various example embodiments of the present disclosure are described in more detail with reference to the accompanying drawings. With respect to reference numerals to elements of each of the drawings, the same elements may be illustrated in other drawings, and like reference numerals may refer to like elements unless stated otherwise. The same or similar elements may be denoted by the same reference numerals even though they are depicted in different drawings. In addition, for the convenience of description, a scale and dimension of each of the elements illustrated in the accompanying drawings may be different from an actual scale and dimension, and thus, embodiments of the present disclosure are not limited to a scale and dimension illustrated in the drawings.

Before starting detailed explanations of figures, components that will be described in the specification are distinguished merely according to functions mainly performed by the components. That is, two or more components which will be described later can be integrated into a single component. Furthermore, a single component which will be explained later can be separated into two or more components. Moreover, each component which will be described can additionally perform some or all of a function executed by another component in addition to the main function thereof. Some or all of the main function of each component which will be explained can be carried out by another component. Accordingly, presence/absence of each component which will be described throughout the specification should be functionally interpreted.

In the conventional mRNA vaccine design, optimization of a 5′ UTR sequence was mainly performed by relying on empirical or iterative experiments, and there were technical limitations in quantitatively predicting in advance the degree of translation efficiency that a specific mRNA sequence would exhibit in actual cells.

The technology described below is a technique for predicting translation efficiency of an mRNA vaccine composed of a specific sequence. The technology described below is a technique for predicting the effect of a secondary structure formed between a 5′ UTR and a coding region initiation site on translation initiation.

Hereinafter, it will be described that a hardware apparatus analyzes the sequence information of an mRNA vaccine to be analyzed to predict in vivo translation efficiency. Hereinafter, an mRNA vaccine sequence to be analyzed is referred to as a candidate mRNA vaccine sequence. The hardware apparatus evaluates whether the candidate mRNA vaccine sequence satisfies a translation efficiency criterion. The hardware apparatus may be implemented in various forms such as a computer device, a PC, a smart device, and a server on a network.

The hardware apparatus predicts the translation efficiency of the candidate mRNA vaccine sequence by using a learning model. The hardware apparatus may select a sequence suitable for actual vaccine manufacture from among a plurality of candidate mRNA sequences. This may reduce unnecessary experimental repetition and significantly reduce the development period and cost of the mRNA vaccine by preventing in advance a sequence having low translation efficiency from being introduced into a manufacturing process.

A learning model refers to a machine learning model. The learning model includes various types of models. For example, a learning model includes a decision tree, a random forest, XGBoost (eXtreme Gradient Boosting), LightGBM (Light Gradient Boosting Machine), CatBoost (Categorical Boosting), KNN (K-nearest neighbor), Naïve Bayes, SVM (support vector machine), and an artificial neural network, and the like.

A DNN (deep neural network) is a representative artificial neural network. A DNN is an artificial neural network model composed of a plurality of hidden layers between an input layer and an output layer. A DNN may model complex non-linear relationships, similarly to a general artificial neural network. Various types of DNN models have been studied. For example, there are CNN (Convolutional Neural Network), RNN (Recurrent Neural Network), RBM (Restricted Boltzmann Machine), DBN (Deep Belief Network), GAN (Generative Adversarial Network), RN (Relation Networks), and the like. Hereinafter, a description will be made focusing on a DNN as a model for predicting translation efficiency of a candidate mRNA vaccine.

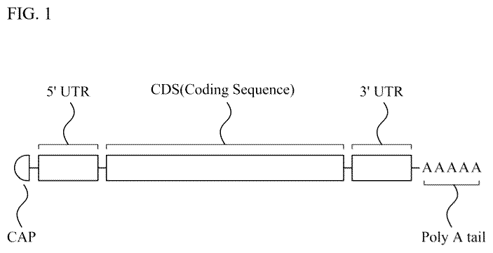

FIG. 1 is an example of an mRNA structure. The mRNA is composed of a cap (CAP), a 5′ UTR, a CDS (Coding sequence), a 3′ UTR, and a poly-A tail in order of the sequence. The cap is located at the 5′ end and helps protein production and protects the mRNA from degradation. The UTR is a non-translated region, and there are a 5′ UTR and a 3′ UTR on either side of the CDS.

The poly-A tail is located at the 3′ end and plays a role in assisting protein production and maintaining stability. The CDS is a sequence used for protein translation and is composed of exons, and includes a start codon and a stop codon. It is known that various translation regulatory mechanisms exist in the 5′ UTR. Therefore, conventional mRNA vaccine research has mainly focused on optimization of the 5′ UTR sequence.

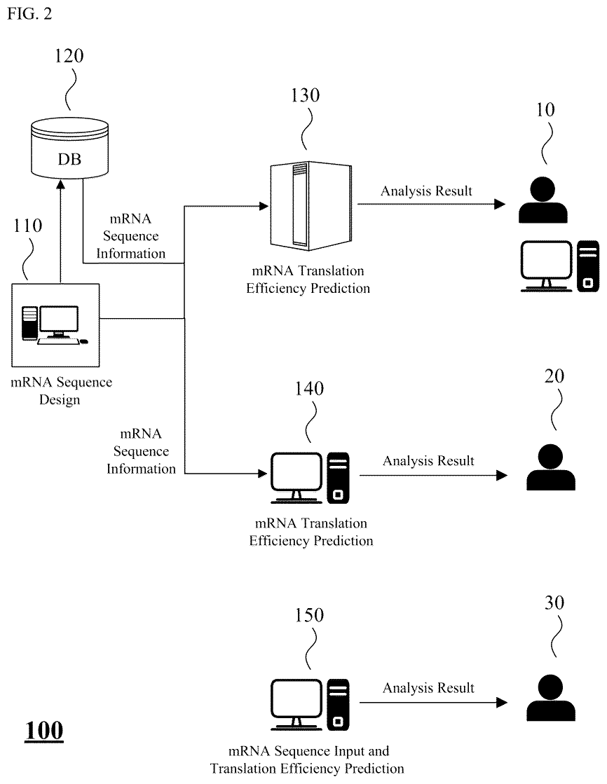

FIG. 2 is an example of a system 100 for predicting translation efficiency of an mRNA vaccine. In FIG. 2, examples are illustrated in which the hardware apparatus is a server 130 and computer terminals 140 and 150.

An mRNA design apparatus 110 designs a specific sequence of a candidate mRNA vaccine. For example, the mRNA design apparatus 110 may receive sequence information for a candidate mRNA vaccine from a developer. Alternatively, the mRNA design apparatus 110 may design a sequence of a candidate mRNA vaccine by using a specific program.

The mRNA design apparatus 110 may design sequence information for a plurality of candidate mRNA vaccines.

The mRNA design apparatus 110 may store sequence information of a plurality of candidate mRNA vaccines in a database (DB) 120.

The server 130 may receive sequence information of a specific candidate mRNA from the mRNA design apparatus 110 or the DB 120. The server 130 may predict whether the specific candidate mRNA may be used as an mRNA vaccine having high translation efficiency. The server 130 may predict translation efficiency based on the sequence information of the specific candidate mRNA. (i) The server 130 may extract at least a portion of sequence information from the sequence information of the specific candidate mRNA. In addition, the server 130 may predict a secondary structure by using the extracted at least a portion of the sequence information. The secondary structure may be a structure formed by the 5′ UTR and another region. (ii) The server 130 may encode the extracted sequence information and secondary structure information and input the encoded information into a pre-trained deep learning model. (iii) The server 130 may predict translation efficiency of the specific candidate mRNA vaccine sequence based on a value output by the deep learning model. (iv) The server 130 may evaluate the validity of the specific candidate mRNA vaccine sequence based on the translation efficiency of the candidate mRNA vaccine sequence. Through this process, the server 130 may evaluate the validity of a plurality of candidate mRNA vaccine sequences.

The input data and model building process used by the deep learning model will be described later. A user 10 may access the server 130 through a user terminal (PC, smartphone, etc.) and check the analysis result performed by the server 130.

A computer terminal 140 may receive sequence information of a specific candidate mRNA from the mRNA design apparatus 110 or the DB 120. The computer terminal 140 may predict whether the specific candidate mRNA may be used as an mRNA vaccine having high translation efficiency. The computer terminal 140 may predict translation efficiency based on the sequence information of the specific candidate mRNA. (i) The computer terminal 140 may extract at least a portion of sequence information from the sequence information of the specific candidate mRNA. In addition, the computer terminal 140 may predict a secondary structure by using the extracted at least a portion of the sequence information. The secondary structure may be a structure formed by the 5′ UTR and another region. (ii) The computer terminal 140 may encode the extracted sequence information and secondary structure information and input the encoded information into a pre-trained deep learning model. (iii) The computer terminal 140 may predict translation efficiency of the specific candidate mRNA vaccine sequence based on a value output by the deep learning model. (iv) The computer terminal 140 may evaluate the validity of the specific candidate mRNA vaccine based on the translation efficiency of the candidate mRNA vaccine sequence. Through this process, the computer terminal 140 may evaluate the validity of a plurality of candidate mRNA vaccine sequences.

A computer terminal 150 corresponds to both a hardware apparatus and an mRNA design apparatus. The computer terminal 150 receives a sequence of a specific candidate mRNA vaccine from a user 30. The computer terminal 150 may predict whether the specific candidate mRNA may be used as an mRNA vaccine having high translation efficiency. (i) The computer terminal 150 may extract at least a portion of sequence information from the sequence information of the specific candidate mRNA. In addition, the computer terminal 150 may predict a secondary structure by using the extracted at least a portion of the sequence information. The secondary structure may be a structure formed by the 5′ UTR and another region. (ii) The computer terminal 150 may encode the extracted sequence information and secondary structure information and input the encoded information into a pre-trained deep learning model. (iii) The computer terminal 150 may predict translation efficiency of the specific candidate mRNA vaccine sequence based on a value output by the deep learning model. (iv) The computer terminal 150 may evaluate the validity of the specific candidate mRNA vaccine based on the translation efficiency of the candidate mRNA vaccine sequence. Through this process, the computer terminal 150 may evaluate the validity of a plurality of candidate mRNA vaccine sequences. The input data and model building process used by the deep learning model will be described later. The user 30 may check the analysis result through the computer terminal 150 used by the user 30.

A deep learning model for predicting translation efficiency of a candidate mRNA will be described. The deep learning model was built using a data set that had been used in research for predicting translation efficiency of conventional mRNA. The conventional model is Optimus 5-Prime from Moderna (Paul J. Sample et al., Human 5′ UTR design and variant effect prediction from a massively parallel translation assay, Nature Biotechnology volume 37, p803-809, 2019, reference). Optimus 5-Prime predicts translation efficiency of a corresponding mRNA based on a 5′ UTR sequence. Optimus 5-Prime is a model generated by using sequences of randomly generated 50 nt-length 5′ UTRs and MRL (Mean Ribosome Load) scores of the corresponding sequences as training data. The deep learning model was built using 280,000 pieces of data used in the development of Optimus 5-Prime. Among the dataset, 20,000 sequences with the highest read counts were used for validation, and the remaining 260,000 sequences were used for training.

The deep learning model predicts translation efficiency using information that is significant for translation efficiency prediction, in addition to 5′ UTR sequence information. In particular, the deep learning model further utilizes a secondary structure formed by the 5′ UTR (or a partial sequence of the 5′ UTR) and a portion of the CDS to predict translation efficiency of the mRNA.

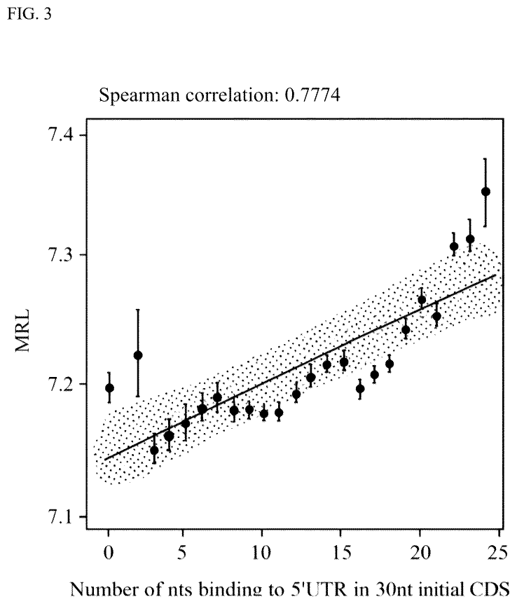

FIG. 3 illustrates a relationship between an interaction of a 5′ UTR and a CDS sequence of an mRNA and translation efficiency. FIG. 3 is a result of analyzing data used in Optimus 5-Prime. FIG. 3 illustrates a relationship between the number of base pairs between the 5′ UTR region and a 30 nt region after the start codon in the CDS of the mRNA and the degree of translation efficiency (MRL score). Referring to FIG. 3, it may be seen that there is a certain correlation between a binding relationship between the 5′ UTR region and the 30 nt region after the start codon in the CDS and translation efficiency. Considering this, the deep learning model uses secondary structure information formed between the 5′ UTR region of the mRNA and the 30 nt region after the start codon in the CDS as input data.

The deep learning model may predict translation efficiency of an mRNA vaccine using input data as shown in Table 1 below.

TABLE 1

| Input data type | Region | Content |
|---|---|---|
| Sequence | 5′ UTR | Fixed 25 nt |
| information | 5′ UTR | 50 nt |
| | CDS | 30 nt after start codon |
| Secondary | 5′ UTR | Binding relationship |
| structure | and CDS | between (i) 25 nt + 50 nt of the |
| information | | 5′ UTR and (ii) 30 nt of the CDS |

The input data includes at least one of \textcircled{1} a fixed 25 nt sequence of the 5′ UTR (primer binding site), \textcircled{2} the last 50 nt of the 5′ UTR (50 nt immediately before the CDS), \textcircled{3} 30 nt after the start codon of the CDS, and \textcircled{4} secondary structure information derived from the 5′ UTR and the CDS region. The secondary structure may be a secondary structure formed by the 5′ UTR (\textcircled{1}+\textcircled{2}) and the CDS (\textcircled{3}). Furthermore, the secondary structure may be a structure formed by a partial sequence of the 5′ UTR and 30 nt of the CDS. The last 50 nt of the 5′ UTR can be designed randomly or by a vaccine designer.

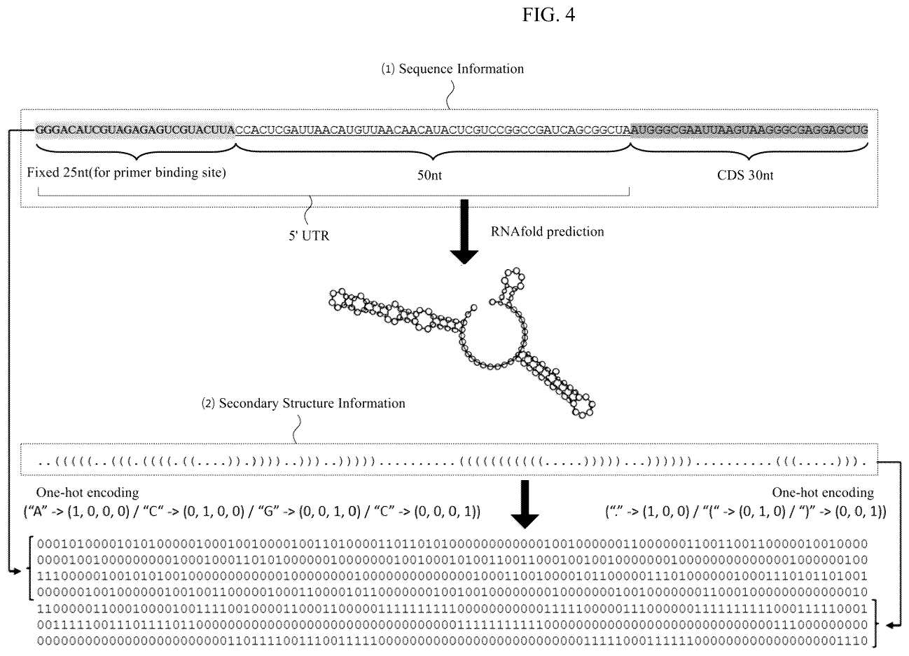

FIG. 4 illustrates an example of input data and an encoding process of a deep learning model. (1) The sequence information includes a fixed 25 nt sequence of the 5′ UTR (primer binding site), a last 50 nt of the 5′ UTR (50 nt immediately before the CDS), and 30 nt after the start codon of the CDS. Accordingly, the sequence information may be an entire 105 nt sequence. (2) The secondary structure information includes binding information formed by the sequence of the 5′ UTR and 30 nt of the CDS. The secondary structure information may be predicted using a publicly available program. For example, the hardware apparatus may calculate secondary structure information for an input sequence (the sequence information of FIG. 4) by using a program such as RNAfold. The secondary structure information includes sequence positions that bind to each other in the entire sequence. In the secondary structure information, corresponding parentheses “(” and “)” indicate bases that bind to each other. The hardware apparatus encodes the input data and inputs the encoded data into the deep learning model. The lower portion of FIG. 4 shows an example of one-hot vector encoding for the input data. Accordingly, the deep learning model may receive a matrix having a size of 105×7.

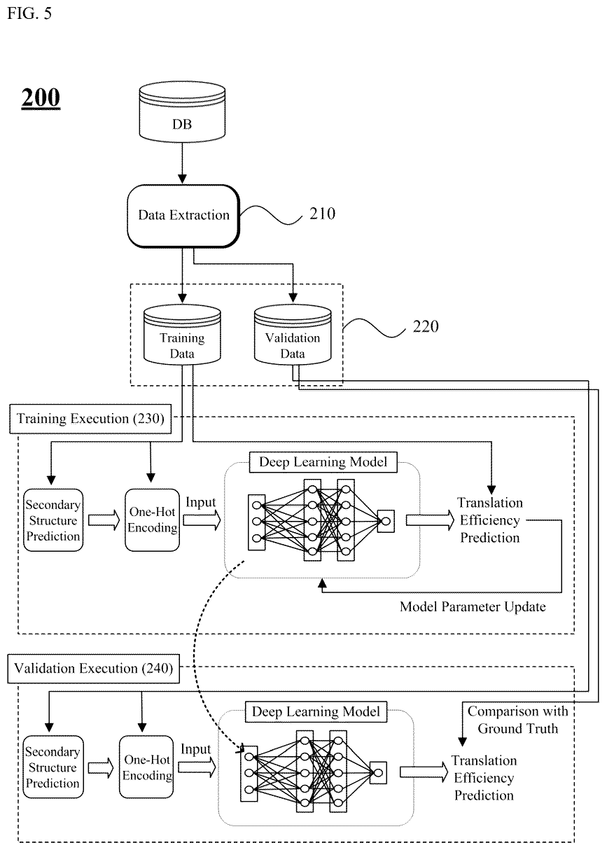

FIG. 5 is an example of a process 200 of building a deep learning model for predicting translation efficiency of a candidate mRNA vaccine. The deep learning model is a model for predicting translation efficiency of an mRNA vaccine. The learning process is described as being performed by a learning apparatus. The learning apparatus refers to a computer device capable of performing data processing and training of a learning model.

The DB may include a data set consisting of entire sequence information of mRNA and translation efficiency information (e.g., MRL scores) of the corresponding mRNA.

The learning apparatus may extract data necessary for model building or validation from the DB (210). The deep learning model uses not only the 5′ UTR of the mRNA but also sequences of various regions and secondary structures formed by a portion of the sequences as input data. Accordingly, the learning apparatus may extract the fixed 25 nt from the 5′ UTR, the last 50 nt from the 5′ UTR, 30 nt after the start codon from the CDS, and MRL scores (=label values) from the data in the DB.

The learning apparatus may divide the entire collected data into training data and validation data.

The learning apparatus performs training of the deep learning model using the training data (220).

The learning apparatus extracts one piece of input data from the training data and performs a learning process. The learning apparatus may predict secondary structure information constituted by the sequence of the 5′ UTR (25 nt+50 nt) and the sequence of the CDS (30 nt). The learning apparatus may predict a secondary structure of an input sequence by using a program such as RNAfold. The learning apparatus may perform one-hot vector encoding of sequence information and secondary structure information of the mRNA as illustrated in FIG. 4. The learning apparatus may predict translation efficiency by inputting encoded input data into the deep learning model.

The learning apparatus updates the parameters of the model so that the deep learning model outputs a value close to a correct answer while comparing a predicted value output by the deep learning model with a label value (e.g., an MRL score of the corresponding sequence). The learning apparatus repeats the learning process using data belonging to the training data.

When the learning process is completed, the learning apparatus may perform validation on the trained deep learning model (230). The learning apparatus extracts one piece of validation data and performs a validation process. The learning apparatus may predict translation efficiency by inputting the selected data into the deep learning model and perform validation by comparing the predicted value with a correct answer value.

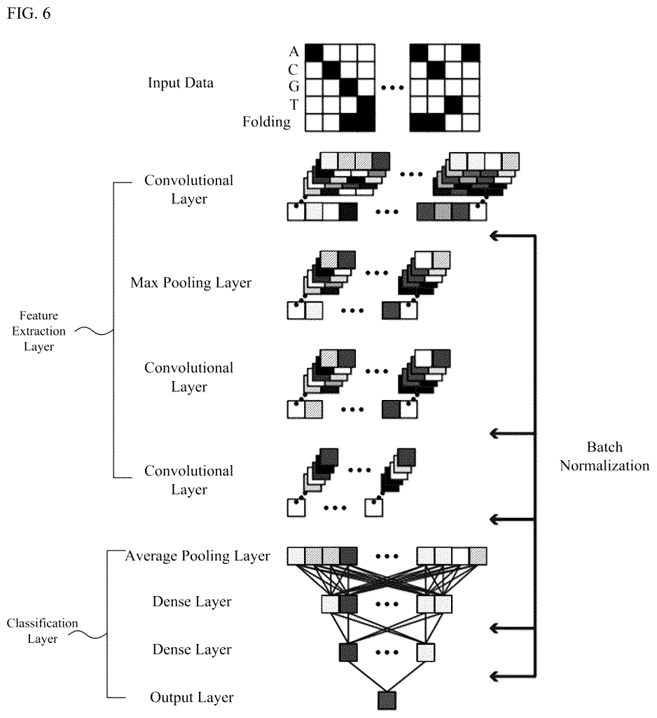

FIG. 6 is an example of a deep learning model for predicting translation efficiency of an mRNA vaccine. The deep learning model may include a feature extraction layer that extracts features from the encoded input data and a classification layer that receives a feature map output from the feature extraction layer and predicts translation efficiency. The feature extraction layer may include a plurality of convolutional layers and max pooling layers. The classification layer may include an average pooling layer, a dense layer, and a final output layer. The output layer may output a final predicted value by using an activation function. In addition, the deep learning model may include batch normalization layers as illustrated in FIG. 6.

The deep learning model was validated the prediction accuracy using 20,000 5′ UTR sequences.

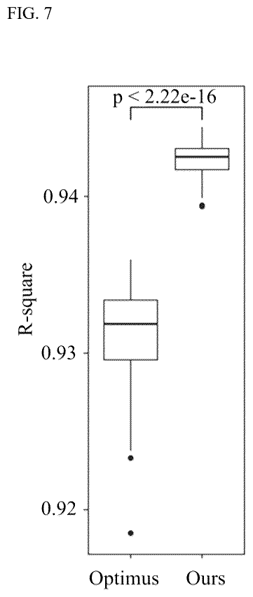

FIG. 7 illustrates a result of evaluating performance of translation efficiency prediction of the built deep learning model. FIG. 7 illustrates translation efficiency prediction performance of the conventional model Optimus 5-prime (indicated as Optimus) and the built deep learning model (indicated as Ours). The performance of translation efficiency was evaluated by a coefficient of determination (R-square). The Optimus 5-prime model has an R-Square value of 0.93, but the built model (Ours) showed an R-Square value of 0.943.

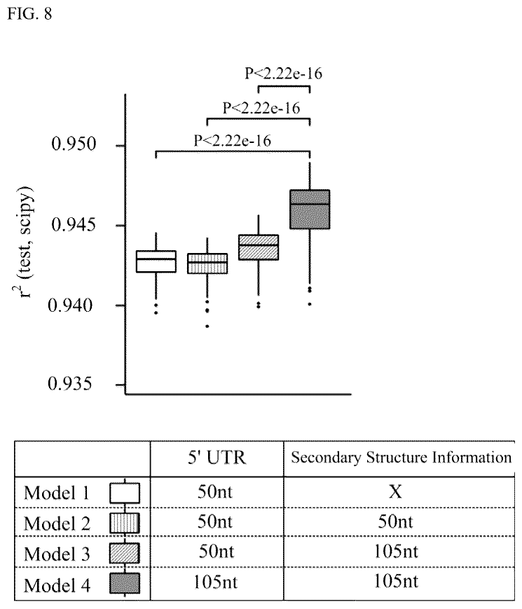

FIG. 8 illustrates a result of comparing performance of deep learning models using various input data. The performance of translation efficiency was evaluated by a coefficient of determination (r²). Researchers evaluated the performance of (i) Model 1 using only the 50 nt of the 5′ UTR as input data, (ii) Model 2 using the 50 nt of the 5′ UTR and secondary structure information formed by the 50 nt region as input data, (iii) Model 3 using the 50 nt of the 5′ UTR and secondary structure information formed by the 105 nt region of the 5′ UTR and the CDS as input data, and (iv) Model 4 using the 105 nt region of the 5′ UTR and the CDS and secondary structure information formed by the 105 nt region as input data. Referring to FIG. 8, Model 3 showed higher performance than Model 1 or Model 2, and Model 4 showed the highest performance. In summary, a deep learning model using secondary structure information together with the 5′ UTR sequence showed higher performance than a model not using secondary structure. In addition, a deep learning model using additional sequences such as the fixed 25 nt and the CDS 30 nt in addition to the 50 nt of the 5′ UTR showed higher performance than a model using only the 50 nt.

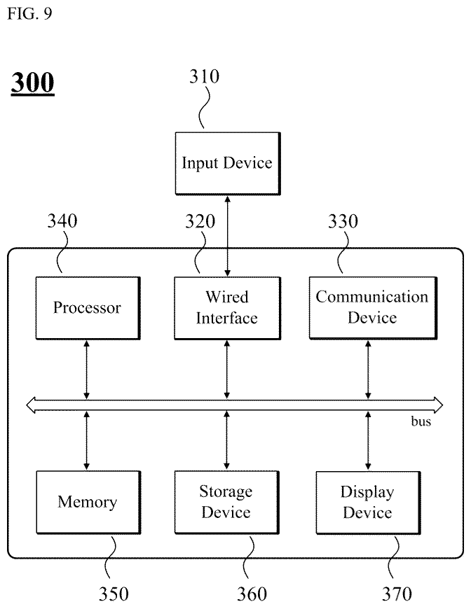

FIG. 9 is an example of a hardware apparatus 300 for predicting translation efficiency of an mRNA vaccine. The hardware apparatus 300 corresponds to the hardware apparatus (130, 140, and 150 of FIG. 2) described above. The hardware apparatus 300 may be physically implemented in various forms. For example, the hardware apparatus 300 may have forms such as a computer device such as a PC, a server of a network, and a dedicated chipset for data processing.

The hardware apparatus 300 may also design a final mRNA sequence using sequence information of an mRNA vaccine having high efficiency.

The hardware apparatus 300 may include an input device 310, a wired interface 320, a communication device 330, a processor 340, a memory 350, and a storage device 360.

Alternatively, the hardware apparatus 300 may include the input device 310, the wired interface 320, the communication device 330, the processor 340, the memory 350, the storage device 360, and a display device 370.

The internal components of the hardware apparatus 300 may be connected by a bus. A specific bus may be used depending on the type of entity being connected. For example, the bus may be any one of AMBA (AHB/AXI/APB), PCIe, SPI (Serial Peripheral Interface), or MIPI (Mobile Industry Processor Interface).

The input device 310 is a device that receives user commands or information.

In addition, the input device 310 may be a device that receives necessary data from a physically connected external device or storage device.

The input device 310 may receive sequence information of a candidate mRNA vaccine.

The input device 310 may be any one of various types of devices. For example, the input device 310 may be at least one of a mouse, a keyboard, a touch input device, a camera, a SCSI (Small Computer System Interface) device, a PCI (Peripheral Component Interconnect) bus-based device, or an ATAPI (ATA Packet Interface) device.

The wired interface 320 is a device component that delivers data transmitted by the input device 310 to the inside of the apparatus. The wired interface 320 may be composed of software drivers and hardware.

The wired interface 320 may include a controller corresponding to each input device, a device driver that controls the operation of the controller, and a kernel I/O subsystem that comprehensively manages input/output control requests of the device driver. The kernel I/O subsystem stores input/output requests from the device driver in a queue and schedules the requests based on request priority or device status.

The wired interface 320 may include interfaces such as PS/2, USB (Universal Serial Bus), an Ethernet port, HDMI, MIPI CSI, DisplayPort, and Thunderbolt.

The wired interface 320 may deliver the sequence information of the candidate mRNA vaccine, translation efficiency of the mRNA vaccine, and the like, to an internal or external entity of the apparatus.

The communication device 330 refers to a component that receives and transmits certain information through an external wired or wireless network. The communication device 330 may be composed of a circuit including an antenna and a communication module (S/W module, chip, etc.) corresponding to a communication protocol. The communication protocol may be at least one of wired LAN (Ethernet), wireless LAN (IEEE 802.11), mobile communication (LTE, 5G NR, etc.), Bluetooth, and NFC.

The communication device 330 may receive sequence information of a candidate mRNA vaccine.

The communication device 330 may transmit the translation efficiency of the mRNA vaccine, which is an analysis result, to an external entity such as a user terminal.

The processor 340 controls operations of overall components of the hardware apparatus 300.

The processor 340 may perform computations on at least one application or computer program for executing a method/operation according to various embodiments of the present disclosure.

The processor 340 is a general-purpose processor that executes at least a portion of a control program installed in the storage device 360 or at least a portion of a program loaded in the memory 350.

The processor 340 may be implemented as circuitry (e.g., processing circuitry) such as a system on chip (SoC) or an integrated circuit (IC).

The processor 340 may include one or more processors. For example, the processor 340 may include a combination of one or more processors such as a CPU (central processing unit), an MPU (microprocessor unit), an MCU (micro controller unit), a GPU (graphic processing unit), an NPU (neural processing unit), a DSP (digital signal processor), an AP (application processor), a CP (communication processor), or any type of processor well known in the art of the present disclosure.

The memory 350 may store data generated in a process of predicting translation efficiency of the candidate mRNA vaccine. The memory 350 is a volatile memory such as a DRAM or an SRAM.

The storage device 360 may store the deep learning model for predicting translation efficiency of an mRNA vaccine. Here, the deep learning model is a pre-trained model.

The storage device 360 may store a program or code for controlling a process of predicting translation efficiency of the candidate mRNA vaccine.

The storage device 360 may store sequence information of the candidate mRNA vaccine. Here, the sequence information may be an entire sequence of a target mRNA.

The storage device 360 may store a program that receives an RNA sequence and predicts a secondary structure derived from the sequence.

The storage device 360 may store analysis results.

The storage device 360 may store sequences of antigen proteins for vaccine design.

The storage device 360 may be implemented as a device such as a hard disk drive, a solid state drive, a USB flash drive, a memory card, an optical disk, or a network-based storage device (Network Attached Storage, cloud storage, etc.).

The display device 370 may output an interface necessary for a data processing process, analysis results, and the like.

The display device 370 may be implemented in various forms of devices.

The display device 370 may be implemented in various display methods such as liquid crystal, plasma, light-emitting diode, organic light-emitting diode, surface-conduction electron-emitter, carbon nano-tube, and nano-crystal.

The processor 340 may extract input data from the sequence information of the candidate mRNA vaccine. The input data may include at least one of \textcircled{1} a fixed 25 nt sequence of the 5′ UTR (primer binding site), \textcircled{2} the last 50 nt of the 5′ UTR (50 nt immediately before the CDS), and \textcircled{3} 30 nt after the start codon of the CDS.

The processor 340 may predict secondary structure information constituted by the sequence extracted as input data. The processor 340 may predict secondary structure information by inputting the input data into a secondary structure prediction program. For example, the processor 340 may predict secondary structure information of 105 nt constituted by the fixed 25 nt sequence of the 5′ UTR, the last 50 nt of the 5′ UTR, and 30 nt after the start codon of the CDS.

The processor 340 performs one-hot vector encoding on the input data, including the sequence and secondary structure information, as illustrated in FIG. 4. Through this, the processor 340 may calculate a vector matrix for the input data.

The processor 340 inputs the encoded input data (or vector matrix) into the trained deep learning model to calculate a predicted value for translation efficiency. Through this, the processor 340 may predict translation efficiency for the candidate mRNA vaccine sequence.

The processor 340 may evaluate the validity of the current candidate mRNA vaccine sequence based on the translation efficiency of the corresponding candidate mRNA vaccine sequence. The processor 340 may select the corresponding candidate mRNA vaccine as a valid mRNA vaccine sequence when the translation efficiency of the corresponding candidate mRNA vaccine sequence is equal to or greater than a predetermined threshold.

The processor 340 may design the mRNA vaccine by linking a sequence of an antigen protein after the start codon of the coding region of the valid mRNA vaccine sequence.

In addition, the processor 340 may link a 3′ UTR (Untranslated Region) for improving stability and translation efficiency of the mRNA to a lower portion of the coding region. The 3′ UTR may be designed to include one or more known stabilizing sequences, followed by addition of a poly(A) tail, thereby completing the overall structure of the mRNA vaccine.

Finally, the mRNA vaccine may be designed to have an overall sequence structure including a 5′ cap structure, a 5′ UTR sequence predicted to have improved translation efficiency, a coding region encoding an antigen protein, a 3′ UTR, and a poly(A) tail.

In addition, the method of predicting translation efficiency of an mRNA vaccine or the method of designing an mRNA vaccine as described above may be implemented as a program (or application) including an executable algorithm that may be executed on a computer. The program may be stored in and provided on a temporary or non-transitory computer readable medium.

The non-transitory computer readable medium refers to a medium that stores data semi-permanently (e.g., the storage device) and is capable of being read by a device, rather than a medium that stores data for a short period of time, such as a register, cache, or memory. Specifically, the various applications or programs described above may be provided by being stored in the non-transitory computer readable medium such as a CD, a DVD, a hard disk, a Blu-ray disk, a USB, a memory card, a read-only memory (ROM), a programmable read only memory (PROM), an erasable PROM (EPROM), an electrically EPROM (EEPROM), or a flash memory.

The transitory computer readable medium refers to various types of RAM such as a static RAM (SRAM), a dynamic RAM (DRAM), a synchronous DRAM (SDRAM), a double data rate SDRAM (DDR SDRAM), an enhanced SDRAM (ESDRAM), a synclink DRAM (SLDRAM), and a direct Rambus RAM (DRRAM).

Various examples and aspects of the present disclosure are described below. These are provided as examples, and do not limit the scope of the present disclosure.

The description herein has been presented to enable any person skilled in the art to make, use and practice the technical features of the present disclosure, and has been provided in the context of one or more particular example applications and their example requirements. Various modifications, additions and substitutions to the described embodiments will be readily apparent to those skilled in the art, and the principles described herein may be applied to other embodiments and applications without departing from the scope of the present disclosure. The description herein and the accompanying drawings provide examples of the technical features of the present disclosure for illustrative purposes. In other words, the disclosed embodiments are intended to illustrate the scope of the technical features of the present disclosure. Thus, the scope of the present disclosure is not limited to the embodiments shown, but is to be accorded the widest scope consistent with the claims. The scope of protection of the present disclosure should be construed based on the following claims, and all technical features within the scope of equivalents thereof should be construed as being included within the scope of the present disclosure.

What is claimed is:

1. A method for designing an mRNA vaccine, the method comprising:
receiving, by a hardware apparatus, sequence information of a candidate mRNA vaccine;
extracting, by the hardware apparatus, a partial sequence from a 5′ UTR (Untranslated Region) in the sequence information;
deriving, by the hardware apparatus, secondary structure information formed by the partial sequence and at least a portion of a sequence after a start codon of a coding region in the sequence information;
predicting, by the hardware apparatus, translation efficiency of the candidate mRNA vaccine by inputting the partial sequence and the secondary structure information into a pre-trained deep learning model;
evaluating, by the hardware apparatus, whether the sequence information of the candidate mRNA vaccine is valid based on the translation efficiency of the candidate mRNA vaccine; and
when the sequence information of the candidate mRNA meets a translation-efficiency threshold, generating, by the hardware apparatus, a final mRNA vaccine sequence by linking a sequence of an antigen protein to the sequence information of the candidate mRNA vaccine.

2. The method of claim 1, wherein the partial sequence includes a 25 nt sequence corresponding to a primer binding site and a 50 nt sequence of the 5′ UTR immediately before a CDS (Coding sequence) region.

3. The method of claim 1, wherein the hardware apparatus further inputs a 30 nt sequence after the start codon of the coding region into the deep learning model.

4. The method of claim 1, wherein the secondary structure information represents a secondary structure formed by a 25 nt sequence to which a primer binds in the 5′ UTR, a 50 nt sequence in the 5′ UTR, and a 30 nt sequence after the start codon of the coding region.

5. The method of claim 1, wherein the hardware apparatus performs one-hot vector encoding on the partial sequence and the secondary structure information and inputs the encoded data into the deep learning model.

6. A method for predicting translation efficiency of an mRNA vaccine, the method comprising:
receiving, by a hardware apparatus, sequence information of a candidate mRNA vaccine;
extracting, by the hardware apparatus, an input sequence including a 25 nt sequence corresponding to a primer binding site in a 5′ UTR (Untranslated Region) of the sequence information, a 50 nt sequence of the 5′ UTR immediately before a CDS (Coding sequence) region, and a 30 nt sequence after a start codon of a coding region;
deriving, by the hardware apparatus, secondary structure information for the input sequence; and
predicting, by the hardware apparatus, translation efficiency of the candidate mRNA vaccine by inputting the input sequence and the secondary structure information into a pre-trained deep learning model.

7. A hardware apparatus for designing an mRNA vaccine, the hardware apparatus comprising:
an interface device configured to receive sequence information of a candidate mRNA vaccine;
a storage device configured to store a pre-trained deep learning model; and
a processor configured to
(i) predict translation efficiency of the candidate mRNA vaccine by inputting a partial sequence of a 5′ UTR (Untranslated Region) of the sequence information and secondary structure information formed by the partial sequence and a sequence after a start codon of a coding region of the sequence information into the deep learning model, and,
(ii) when the translation efficiency of the candidate mRNA vaccine meets a translation-efficiency threshold, generate a final mRNA vaccine sequence by linking a sequence of an antigen protein to the sequence information of the candidate mRNA vaccine.

8. The hardware apparatus of claim 7, wherein the partial sequence includes a 25 nt sequence corresponding to a primer binding site and a 50 nt sequence of the 5′ UTR immediately before a CDS (Coding sequence) region.

9. The hardware apparatus of claim 7, wherein the processor further inputs a 30 nt sequence after the start codon of the coding region into the deep learning model.

10. The hardware apparatus of claim 7, wherein the secondary structure information represents a secondary structure formed by a 25 nt sequence to which a primer binds in the 5′ UTR, a selected 50 nt sequence in the 5′ UTR, and a 30 nt sequence after the start codon of the coding region.
